SEQUENCE LISTING (5 sequences)

SEQ ID NO: 1 - protein, 358 aa, Homo sapiens
MAEGERQPPPDSSEEAPPATQNFIIPKKEIHTVPDMGKWKRSQAYADYIGFILTLNEGVKGKKLTFEYRVSEMWNEVHEEKEQAAKQSVSCDECIPLPRAGHCAPSEAIEKLVALLNTLDRWIDETPPVDQPSRFGNKAYRTWYAKLDEEAENLVATVVPTHLAAAVPEVAVYLKESVGNSTRIDYGTGHEAAFAAFLCCLCKIGVLRVDDQIAIVFKVFNRYLEVMRKLQKTYRMEPAGSQGVWGLDDFQFLPFIWGSSQLIDHPYLEPRHFVDEKAVNENHKDYMFLECILFITEMKTGPFAEHSNQLWNISAVPSWSKVNQGLIRMYKAECLEKFPVIQHFKFGSLLPIHPVTSG

SEQ ID NO: 2 - protein, 323 aa, Xenopus laevis
MAEKEQNTDHAEDEIPPSLVGHTFIVPKKEINMVPDMGKWKRSQAYADYIGFVLALNEAVKGKKLTDDYTVSEVIHKLMALLDTLDRWIDETPPMDQPSRFGNKAFRTWYARLDKEAESLVSTVIPVHLSAAVPEVALYLKESVGNSTRIDYGTGHEAAFAAFLCCLCKIGVLKVDDQLAIVFRVFNRYLEVMRKLQKTYRMEPAGSQGVWGLDDFQFLPFIWGSAQLVDHSTLEPRHFVDEKIVNENHKDYMFLECILFITEMKTGPFAEHSNQLWNISAVPAWSKVNQGLIRMYKAECLEKFPVIQHFKFGSLLPIQPVKS

SEQ ID NO: 3 - protein, 398 aa, Drosophila melanogaster
MASGINQAAGKLPAIAKKVQNLGDMGVWQKSRAFHDLIGYINGTSSAIQGIKTTDEIFESEMLKKLLRLFDALEKLVEQNPPLEQPQRFGNKAYRDWAQAMRELLPELLEQLLPDDKKRYQVELGQYLTESFGNATRIDYGTGHELSFLFFLCSLFKAEILQERDIVSAALRLFNRYLELARQLQRTYNMEPAGSQGVWSLDDFQFVPFIWGSAQLAVKSPFDPSKFVDEAIITEYKDHFMFISCIDYICKVKTGHFGEHSNQLWSITDVPTWAKINAGLVKMYQKEILSKFPVIQHVYFGELMTFEPVSSGTTLSNARLGHVAPPPSKRICIGTPNLVPPVPVATAPPPPAESLSIEQNVGDSSSESSDNSVVLRPSTSSSSLVAAAEGSGDKPSKE

SEQ ID NO: 4 - protein, 375 aa, Caenorhabditis elegans
MAENSYKPPEKMIKNVFDLNPWYFSKAYEEYLAFLHRLNDSVVGVHTTADMRCTDLVISFIDMLDKLEKWAEEIPLEDVSEQRFGNKAYRKFYEKLCKESPDLLASVLPENVHDALVELVPYFTESFGNATRIDYGSGHEANFLILLFCLQKLGVFTENDDKVLVLRIFNKYLRVCRHLQTRFKMEPAGSRGVHAIDDFQFAPFIFGSAQLIGSKSIVPDSYLKKNIVETHAHTSLFLDCVNFINQTKTGPFHEHSNQLWNISAVPHWKKVNSGMFKMYEGEVLKKFPVVQHMMFGSLFSFDRSEHPRESMEDAPSQDPMPPRAPAQHGMFAFKIPSAANTSTVHSSSVVESGDLRRLHSEKHPNEHCPPPMADA

SEQ ID NO: 5 - protein, 396 aa, Saccharomyces cerevisiae
MSLDRVDWPHATFSTPVKRIFDTQTTLDFQSSLAIHRIKYHLHKYTTLISHCSDPDPHATASSIAMVNGLMGVLDKLAHLIDETPPLPGPRRYGNLACREWHHKLDERLPQWLQEMLPSEYHEVVPELQYYLGNSFGSSTRLDYGTGHELSFMATVAALDMLGMFPHMRGADVFLLFNKYYTIMRRLILTYTLEPAGSHGVWGLDDHFHLVYILGSSQWQLLDAQAPLQPREILDKSLVREYKDTNFYCQGINFINEVKMGPFEEHSPILYDIAVTVPRWSKVCKGLLKMYSVEVLKKFPVVQHFWFGTGFFPWVNIQNGTDLPVFEEKEEESIEQANAGSPGREQTSTRFPTSTSMPPPGVPPSGNNINYLLSHQNQSHRNQTSFSRDRLRRVVV

(19) United States
(12) Patent Application Publication
(10) Pub. No.: US 2008/0153773 A1
(43) Pub. Date: Jun. 26, 2008

(54) MODULATORS OF PHOSPHOTYROSYL PHOSPHATASE ACTIVATOR

(76) Inventor: Yigong Shi, Plainsboro, NJ (US)

Correspondence Address: PEPPER HAMILTON LLP, ONE MELLON CENTER, 50TH FLOOR, 500 GRANT STREET, PITTSBURGH, PA, 15219, US

(21) Appl. No.: 11/834,386

(22) Filed: Aug. 6, 2007

Related U.S. Application Data
(60) Provisional application No. 60/836,302, filed on Aug. 8, 2006.

(51) Int. Cl. A61K 31/70; C07H 19/04; G06G 7/48

(52) U.S. Cl. 514/47; 536/26.3; 703/11

(57) ABSTRACT

Atomic coordinates for human phosphotyrosyl phosphatase activator (PTPA) and ATPγS bound by PTPA, as well as methods for using these atomic coordinates to prepare ATPase inhibitors of PTPA and ATPase inhibitors prepared using such methods are provided herein. Comprehensive biochemical analyses of the interactions of PTPA with ATP and protein phosphatase 2A are also provided. Compositions including mimetics and small molecules of the invention and, optionally, secondary agents may be used to treat disorders in which PTPA ATPase activity plays a contributing role.

### CROSS-REFERENCE TO RELATED APPLICATIONS

This application claims priority to and benefit of U.S. Provisional Application No. 60/836,302 filed on Aug. 8, 2006; the entire contents of which are hereby incorporated by reference in its entirety.

### GOVERNMENT INTERESTS

Not applicable

### PARTIES TO A JOINT RESEARCH AGREEMENT

Not applicable

### INCORPORATION BY REFERENCE OF MATERIAL SUBMITTED ON A COMPACT DISC

Not applicable

### BACKGROUND

1. Field of Invention

The invention presented herein provides compositions and methods for modulation of phosphotyrosyl phosphatase activator.

2. Description of Related Art

Protein phosphatase 2A (PP2A) is a highly conserved Ser/Thr phosphatase involved in many aspects of cellular activity, and has been shown to be an important tumor suppressor protein. Despite its importance, PP2A function is poorly understood which is at least in part due to the complex composition of PP2A and its mechanisms for regulation.

PP2A is made up of at least three subunits. The core component of PP2A is made up of a catalytic (C) subunit and a scaffold protein (A or PR65) subunit. The PP2A core component interacts with a third regulatory (B) subunit which determines substrate specificity as well as the spatial and temporal functions of PP2A to form a hetero-trimeric holoenzyme. B subunits have been separated into four subfamilies. B (or PR55), B′ (or B56 or PR61), B″ (or PR72), and B′″ (or PR93/PR110), with at least 16 members in each subfamily. The expression level of various types of B subunits is highly diverse depending upon cell types and tissues.

In addition to its Ser/Thr phosphatase activity, PP2A exhibits a basal level of phosphotyrosyl phosphatase activity which is stimulated by phosphotyrosyl phosphatase activator (PTPA), a protein with no sequence homology to any known protein. Activation of the phosphotyrosyl activity of PP2A by PTPA has significant influence over control of cellular signaling and produces a number of effects on the cell. PTPA has been shown to play an essential role in survival in mammalian cells. However, how PTPA performs this function has remained unclear. For example, although ATP and magnesium were required for PTPA activity on PP2A, ATP hydrolysis was not thought to be a contributing factor to this modulatory activity. Additionally, PTPA was recently shown to reactivate an inactive PP2A population that is associated with a methyl esterase by a mechanism that does not appear to rely on the ATPase activity of the PTPA. This aspect of PTPA function has gained considerable visibility prompting the name of PTPA to be re-interpreted as “PP2A phosphatase activator.” PTPA appears to modulate phosphotyrosyl and Ser/Thr phosphatase activity in PP2A by performing a peptidyl-proline cis-trans isomerase that targets Pro190 in the catalytic subunit of PP2A, an action that appears to requires ATPase activity in vitro. However, PTPA lacks signature sequence motifs that are common to other known classes of ATPases.

The function of the observed ATPase activity of PTPA and the mechanism for PTPA ATPase activation are provided herein as are the atomic coordinates for free PTPA and PTPA/ATP complex. These results provide the basis for generating small molecules and mimetics that can be used to inactivate PTPA ATPase activity, thereby inactivating PTPA and inhibiting phosphotyrosyl activity of PP2A which may be used as agents for treating various disorders in which PTPA may play a contributing role.

### BRIEF SUMMARY OF THE INVENTION

Embodiments of the invention presented herein are generally directed to PTPA binding compounds including a molecule having a three-dimensional structure corresponding to the atomic coordinates of at least a portion of ATPγS bound to PTPA. In various embodiments, the molecule may binds to PTPA and be an ATPase inhibitor of PTPA. In certain embodiments, the molecule may bind to a pocket formed by β2, α6, β3, α9 and α17 of PTPA.

In some embodiments, the molecule may be a non-hydrolyzable ATP analogue, and in other embodiments, the molecule may include adenine and ribose moieties of ATP. In still other embodiments, the molecule may be selected from mimetics and analogs of adenosine or the molecule may be selected from mimetics and analogs of adenine. In various embodiments, the molecule may bind to PTPA with a greater affinity than ATP and may inhibit modulation of PP2A by PTPA. In certain embodiments, the molecule may inhibit tyrosine phosphorylation catalyzed by PP2A. In particular embodiments, the compound may further include a pharmaceutically acceptable excipient or carrier.

Some embodiments of the invention include a pharmaceutical composition including an effective amount of a compound that mimics the structure of at least a portion of ATPγS bound to PPTA and a pharmaceutically acceptable excipient or carrier.

Other embodiments of the invention include methods for preparing a PTPA binding compound including the steps of applying a three-dimensional molecular modeling algorithm to the atomic coordinates of ATPβS bound to PTPA, determining spatial coordinates of the ATPγS, electronically screening stored spatial coordinates of candidate compounds against the spatial coordinates of the ATPγS and identifying compounds that mimic the structure of the ATPγS bound to PPTA.

In particular embodiments the method may further include identifying candidate compounds that deviate from the atomic coordinates of the ATPγS bound by PTPA by a root mean square deviation of less than about 10 angstroms, and in various embodiments, the method may further include the step of testing the identified compounds for binding PTPA. In some embodiments, the method may include testing the identified compounds for binding to a pocket formed by β2, α6, β3, α9 and α17 of PTPA, and in others, the method may include testing the identified compound for inhibiting ATPase activity of PTPA in the presence of PP2A. In still other embodiments, the method may include identifying a compound that binds to PTPA with a greater affinity than ATP. In certain embodiments, the method may include the step of identifying compounds that inhibit modulation of PP2A by PTPA and, in certain others, inhibit PTPA stimulated tyrosine phosphorylation activity catalyzed by PP2A.

Other embodiments of the invention include a pharmaceutical composition including an effective amount of a compound prepared by the method including applying a three-dimensional molecular modeling algorithm to the atomic coordinates of ATPγS bound to PTPA, determining spatial coordinates of the ATPγS, electronically screening stored spatial coordinates of candidate compounds against the spatial coordinates of the ATPγS and identifying compounds that mimic the structure of the ATPγS bound to PTPA, and a pharmaceutically effective excipient or carrier.

Still other embodiments of the invention include a compound including a molecule substantially complementary to a pocket formed by β2, α6, β3, α9 and α17 of PTPA. IN various embodiments, the molecule may bind to PTPA and in certain embodiments the molecule may bind to a pocket formed by β2, α6, β3, α9 and α17 of PTPA. In some embodiments, the molecule may be an ATPase inhibitor of PTPA, and in particular embodiments, the molecule is a non-hydrolyzable ATP analogue. In some embodiments, the molecule may have a shape, a charge distribution, a size or combinations thereof substantially complementary to the pocket, and in others, the molecule may include adenine and ribose moieties of ATP. In still other embodiments, the molecule may be selected from mimetics and analogs of adenosine, or the molecule may be selected from mimetics and analogs of adenine. In certain embodiments, the molecule may bind to PTPA with a greater affinity than ATP and in certain others, the molecule may inhibit modulation of PP2A by PTPA. In particular embodiments, the molecule may inhibit tyrosine phosphorylation catalyzed by PP2A. In yet other embodiments, the compound may further include a pharmaceutically acceptable excipient or carrier.

Yet other embodiments of the invention include a method for preparing a PTPA binding compound including the steps of applying a three-dimensional molecular modeling algorithm to the atomic coordinates of PTPA, determining spatial coordinates of a pocket formed by β2, α6, β3, α9 and α17 of PTPA, electronically screening stored spatial coordinates of candidate compounds against the spatial coordinates of the pocket and identifying compounds that are substantially complementary to the pocket. In some embodiments, the method may further include the step of testing the identified compounds for binding PTPA and in others the method may include the step of testing the identified compounds for binding to a pocket formed by β2, α6, β3, α9 and α17 of PTPA. In still others, the method may include the step of testing the identified compound for inhibiting ATPase activity of PTPA in the presence of PP2A. In certain embodiments, the method may include identifying compounds that bind to PTPA with a greater affinity than ATP and, in certain others, the step of identifying compounds that inhibit modulation of PP2A by PTPA. In particular embodiments, the method may include the step of identifying compounds that inhibit PTPA stimulated tyrosine phosphorylation activity catalyzed by PP2A.

### DESCRIPTION OF DRAWINGS

For a fuller understanding of the nature and advantages of the present invention reference should be made to the following detailed description taken in connection with the accompanying drawings. The file of this patent contains at least one drawing/photograph executed in color. Copies of this patent with color drawing(s)/photograph(s) will be provided to the USPTO upon request and payment of the necessary fee. All figures where structural representations are shown were prepared using MOLSCRIPT (Kraulis (1991) J Appl Cystallogr 24:946-950) and GRASP (Nicholls et al. (1991) Proteins: Struct Funct Genet 11:281-296).

FIG. 1A shows the structure of human phosphotyrosyl phosphatase activator (PTPA) including three-subdomains: core (blue), linker (green), and lid (magenta).

FIG. 1B shows the surface potential of human PTPA.

FIG. 2 shows a sequence alignment of PTPA across five species: Homo sapiens (hs), Xenopus laevis (xl), Drosophila melanogaster (dm), Caenorhabditis elegans (ce), and Saccharomyces cerevisiae (sc).

FIG. 3A shows a wire diagram of PTPA highlighting invariant residues.

FIG. 3B shows a surface map of PTPA highlighting invariant residues.

FIG. 4A shows a representative SDS-PAGE gels stained by coomassie blue illustrating the results of a GST-mediated pull-down assay

FIG. 4B shows a ribbon diagram of PTPA highlighting the five loss-of-function mutations that cause loss of binding of PTPA to PP2A that affect amino acids in the same surface area of the PTPA structure.

FIG. 4C shows a representative native PAGE illustrating interaction between PTPA and PP2A A-C dimer.

FIG. 5A shows that neither PTPA nor PP2A alone exhibited any detectable ATPase activity, and incubation of PP2A A-C dimer with PTPA in the presence of ATP/Mg²⁺ led to a significant ATPase activity that is inhibited by okadaic acid.

FIG. 5B shows the Km value determined using an equi-molar amount of PP2A and PTPA.

FIG. 5C shows the effect of a number of PTPA mutations on ATPase activity in the presence of PP2A.

FIG. 5D shows a ribbon diagram of PTPA highlighting the residues whose mutation led to compromised ATPase activity.

FIG. 5E shows a ribbon diagram of the structure of the ATPγS-bound PTPA (green and light green) compared with free PTPA (blue and light blue).

FIG. 5F shows a stereo diagram of the bound ATPγS and surrounding region.

FIG. 6A shows the effect of PTPA concentration on pTyr activity of PP2A in the presence and absence of ATP/Mg²⁺.

FIG. 6B shows the effect of ATPγS on the pTyr activity PP2A in the presence and absence of PTPA.

FIG. 6C shows the effect of a number of PTPA mutations on the pTyr activity of PP2A.

FIG. 6D shows the effect of PTPA and various PTPA mutants on the pSer/pThr phosphatase of PP2A.

FIG. 6E shows the effect of various concentrations of PTPA and PTPA G290D on the pSer/pThr activity of PP2A.

FIG. 6F shows the effect of PTPA and various PTPA mutants on the substrate specificity PP2A.

FIG. 6G shows the effect of a number of PTPA mutations on the pTyr activity of PP2A using a fluorescently labeled substrate.

FIG. 6H shows a working model for PTPA on the regulation of PP2A function.

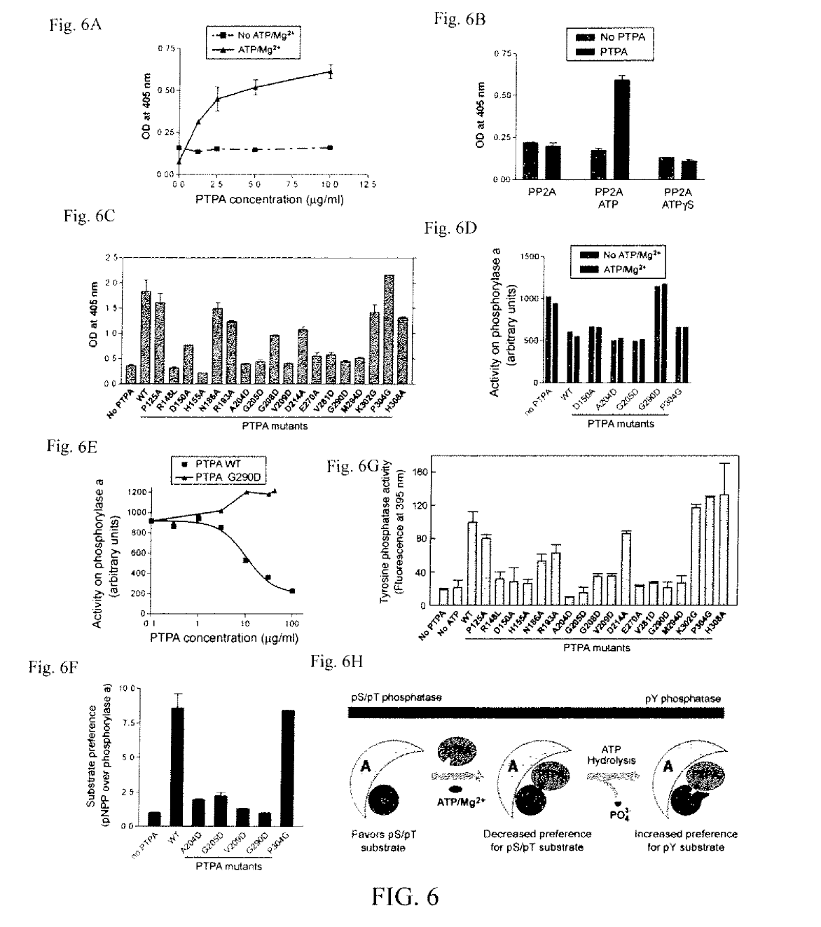

All error bars shown in this figure and FIG. 6 are standard deviations over three independent experiments.

### DETAILED DESCRIPTION

It must be noted that, as used herein and in the appended claims, the singular forms “a,” “an,” and “the” include plural reference unless the context clearly dictates otherwise. Unless defined otherwise, all technical and scientific terms used herein, have the same meanings as commonly understood by one of ordinary skill in the art. Although any methods similar or equivalent to those described herein can be used in the practice or testing of embodiments of the present invention, the preferred methods are now described. All publications and references mentioned herein are incorporated by reference. Nothing herein is to be construed as an admission that the invention is not entitled to antedate such disclosure by virtue of prior invention.

As used herein, the term “about” means plus or minus 10% of the numerical value of the number with which it is being used. Therefore, about 50% means in the range of 45%-55%.

The terms “mimetic,” “peptide mimetic,” and “peptidomimetic” are used interchangeably herein, and generally refer to a peptide, partial peptide or non-peptide molecule that mimics the tertiary binding structure or activity of a selected native peptide or protein functional domain (e.g., binding motif or active site). These peptide mimetics include recombinantly or chemically produced peptides, recombinantly or chemically modified peptides, as well as non-peptide agents, such as small molecule drug mimetics as further described below. Mimetic compounds can have additional characteristics that enhance their therapeutic application, such as increased cell permeability, greater affinity and/or avidity, and prolonged biological half-life.

As used herein, the terms “pharmaceutically acceptable,” “physiologically tolerable,” grammatical variations thereof, as they refer to compositions, carriers, diluents, and reagents, are used interchangeably and represent that the materials are capable of administration upon a mammal without the production of undesirable physiological effects such as nausea, dizziness, rash, or gastric upset.

“Providing,” when used in conjunction with a therapeutic, means to administer a therapeutic directly into or onto a target tissue, or to administer a therapeutic to a patient whereby the therapeutic positively impacts the tissue to which it is targeted.

As used herein, “subject,” “patient” or “individual” refers to an animal or mammal including, but not limited to, a human, dog, cat, horse, cow, pig, sheep, goat, chicken, monkey, rabbit, rat, or mouse, etc.

As used herein, the term “therapeutic” means an agent utilized to treat, combat, ameliorate, prevent or improve an unwanted condition or disease of a patient. Embodiments of the present invention are directed to promote apoptosis and, thus, cell death.

The terms “therapeutically effective amount” or “effective amount,” as used herein, may be used interchangeably and refer to an amount of a therapeutic compound component of the present invention. For example, a therapeutically effective amount of a therapeutic compound is a predetermined amount calculated to achieve the desired effect, i.e., to effectively modulate the activity of a protein phosphorylase.

“Inhibitor” means a compound which reduces or prevents a particular interaction or reaction. For example, the binding of an non-hydrolyzable agent to the site of ATP hydrolysis of phosphotyrosyl phosphatase activator (PTPA) may inhibit the ATP hydrolysis associated with PTPA thereby inhibiting phosphotyrosyl activity of protein phosphatase 2A (PP2A).

“Pharmaceutically acceptable salts” include both acid and base addition salts. “Pharmaceutically acceptable acid addition salt” refers to those salts which retain the biological effectiveness and properties of the free bases and which are not biologically or otherwise undesirable and formed with inorganic acids, such as hydrochloric acid, hydrobromic acid, sulfuric acid, nitric acid, carbonic acid, phosphoric acid, and the like. Organic acids may be selected from aliphatic, cycloaliphatic, aromatic, araliphatic, heterocyclic, carboxylic, and sulfonic classes of organic acids, such as formic acid, acetic acid, propionic acid, glycolic acid, gluconic acid, lactic acid, pyruvic acid, oxalic acid, malic acid, maleic acid, maloneic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, p-toluenesulfonic acid, salicyclic acid, and the like.

The invention described herein is generally directed to atomic coordinates defining phosphotyrosyl phosphatase activator (PTPA), methods for using the atomic coordinates of PTPA, mimetics and small molecules prepared using such methods, and pharmaceutical compositions made from mimetics and small molecules so prepared.

The atomic coordinates of various embodiments of the invention were derived from a truncated version of PTPA (residues 19-323) that retained its full protein phosphatase 2A (PP2A) phosphotyrosyl phosphatase activating activity and was cloned, purified, and crystallized. The structure of the truncated PTPA was determined using molecular replacement and refined the atomic model to 1.9 Å resolution using the atomic coordinates of a previously described PTPA structure in the Protein Data Bank (accession code 2G62) (Table 1). Two molecules of PTPA are in each asymmetric unit, which can be superimposed with a root-mean-squared deviation (RMSD) of about 1.27 Å for all aligned Cα atoms. Since these two molecules share all features important for discussion, we focus our attention on one such molecule.

TABLE 1
Data Collection and Statistics from Crystallographic Analysis

| Protein | PTPA | PTPA + ATPγS |
|---|---|---|
| Beamline | NSLS-X29 | NSLS-X29 |
| Space group | C2 | P1 |
| Resolution (Å) | 99.0-1.9 Å | 100.0-2.5 Å |
| Total observations | 108,795 | 138,863 |
| Unique observations | 36,825 | 76,255 |
| Data coverage (outer shell) | 95.4% (94.6%) | 94.6% (91.1%) |
| R_{sym} (outer shell) | 0.056 (0.313) | 0.083 (0.377) |

Refinement

| | | |
|---|---|---|
| Resolution range (Å) | 50.0-1.9 Å) | 50.0-2.5 Å |
| Number of reflections (\|F\| > 0) | 35,226 | 70,625 |
| Data Coverage | 92.2% | 92.8% |
| R_{working}/R_{free} | 0.183/0.239 | 0.249/0.312 |
| Number of Atoms | 5,040 | 19,620 |
| Number of waters | 205 | 172 |
| RMSD bond length (Å) | 0.008 | 0.011 |
| RMSD bond angles (deg) | 1.37 | 1.27 |

Ramachandran Plot

| | | |
|---|---|---|
| Most favored (%) | 91.3 | 90.1 |
| Additionally allowed (%) | 8.1 | 9.6 |
| Generously allowed (%) | 0.6 | 0.2 |
| Disallowed (%) | 0.0 | 0.1 |

R_{sym} = ΣₕΣₗ|I_{h,l} − Iₕ|/ΣₕΣₗ I_{h,l}, where Iₕ is the mean intensity of the i observations of symmetry-related reflections of h.
R = Σ|F_{obs} − F_{calc}|/ΣF_{obs}, where F_{obs} = Fₚ, and F_{calc} is the calculated protein structure factor from the atomic model (R_{free} was calculated with 5% of the reflections). RMSD in bond lengths and angles is the deviation from ideal values, and the RMSD in B factors is calculated between bonded atoms.

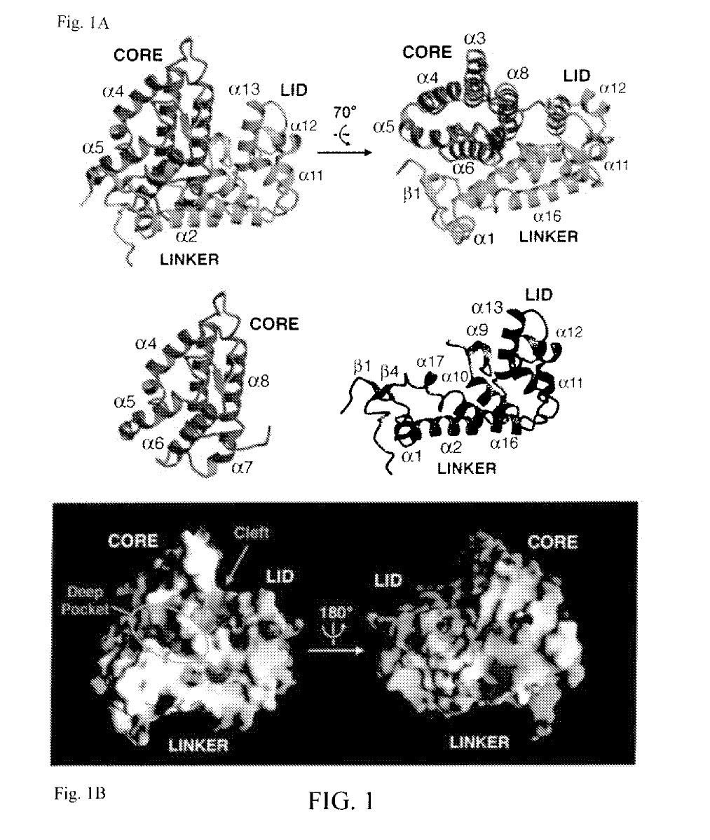

The structure for the truncated PTPA is provided in FIG. 1. FIG. 1A shows the ribbon structure of PTPA from two angles (upper panel) and separated by subdomain (lower panel). As can be seen by the ribbon structure, PTPA adopts a mostly α-helical, compact structure, with 17 α helices and four short β strands (selected secondary structural elements are labeled in each panel). The structure appears to contain three subdomains: core (blue), linker (green), and lid (magenta). The core domain includes two short β strands (β2 and β3) and 6 contiguous α helices (α3-α8) arranged in two layers. The outer layer has α3 and α4; the inner layer which is in contact with the other two domains includes α5, α6, α7, and α8 (FIG. 1A). The C-terminal lid domain includes 5 contiguous α helices (α11-α15) which are connected to the core domain via the linker domain. As the name suggests, the linker domain contains sequence elements from the N-terminal region (α1, α2, and β1), the center (α9 and α10), and the C-terminus (α16, α17, and β4). The extended linker domain joins the compact core and lid domains. It is of note that a large cleft exists between the lid and core domains opposite the linker domain.

FIG. 1B shows the surface potential of PTPA. The positively and negatively charged surface areas are colored blue and red, respectively. FIG. 1B shows several acidic residues located at the bottom of the cleft between the lid and core domains indicated by the green arrow. In addition, a deep, negatively charged pocket (circled in green) is observed between the core and the linker domains.

In general, the structure of PTPA appears to represent a previously unreported protein fold. In fact, a search of the Protein Data Bank using the program DALI did not yield any entry with significant structural similarity. Several entries showed limited homology with select regions of PTPA; however, none of these entries is an ATPase or peptidyl-proline isomerase. Therefore, PTPA may represent a new family of proteins having ATPase and peptidyl-proline isomerase activity.

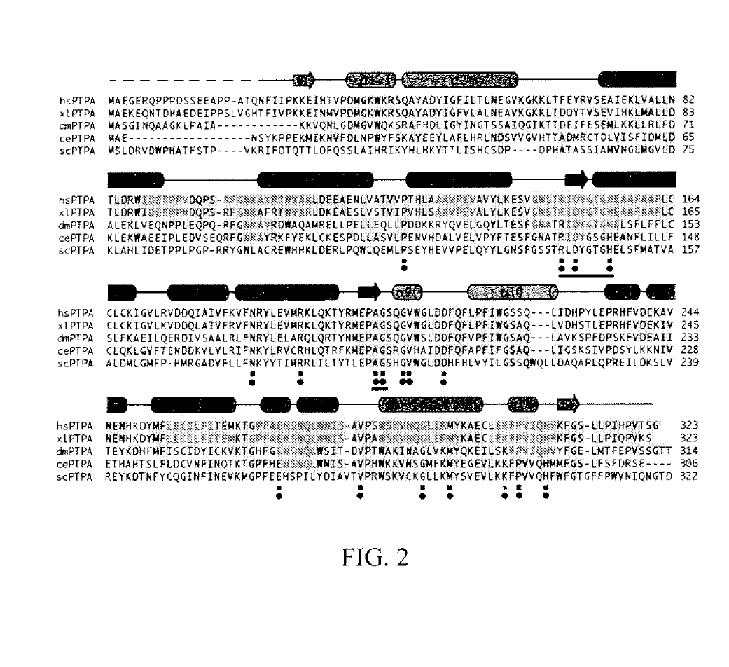

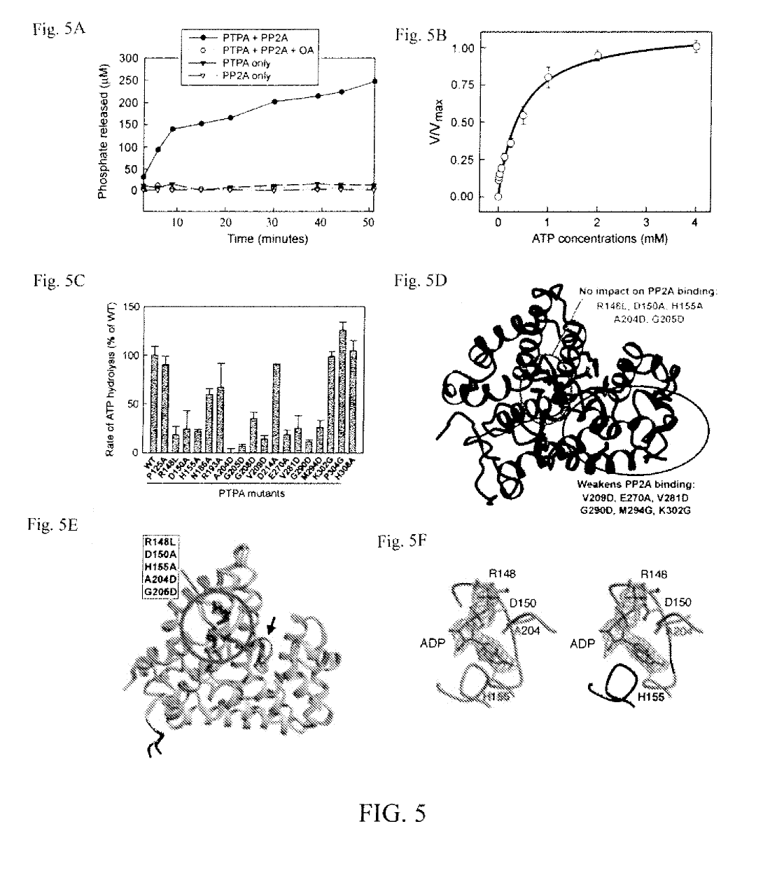

PTPA is highly conserved across species, and the functions of PTPA are also conserved among PTPA homologs across species. This functional conservation may be supported by shared structural features on the surface of PTPA. FIG. 2 shows a sequence alignment of PTPA across species: Homo sapiens (hs), Xenopus laevis (xl), Drosophila melanogaster (dm), Caenorhabditis elegans (ce), and Saccharomyces cerevisiae (se). The secondary structure elements of PTPA provided by the three-dimensional crystal structure described above is shown across the top of the sequence alignment. Color coding of the secondary structural elements is the same as in FIG. 1A and shows the described subdomains. Conserved amino acids are highlighted in yellow. The effects of mutation in PP2A binding (squares) and ATPase activity (circles) are presented below the sequence alignment with the colors indicating the severity of the mutations: red signifies loss of function, orange signifies partial loss of function, and green represents no significant effect. The blue line below the squares and circles show the amino acid residues that are close to ATPγS in the crystal structure of ATPγS-bound PTPA (FIG. 5F). 68 amino acids were found invariant across the five representative species compared in FIG. 2. Of these, 39 are fully or partially exposed to solvent.

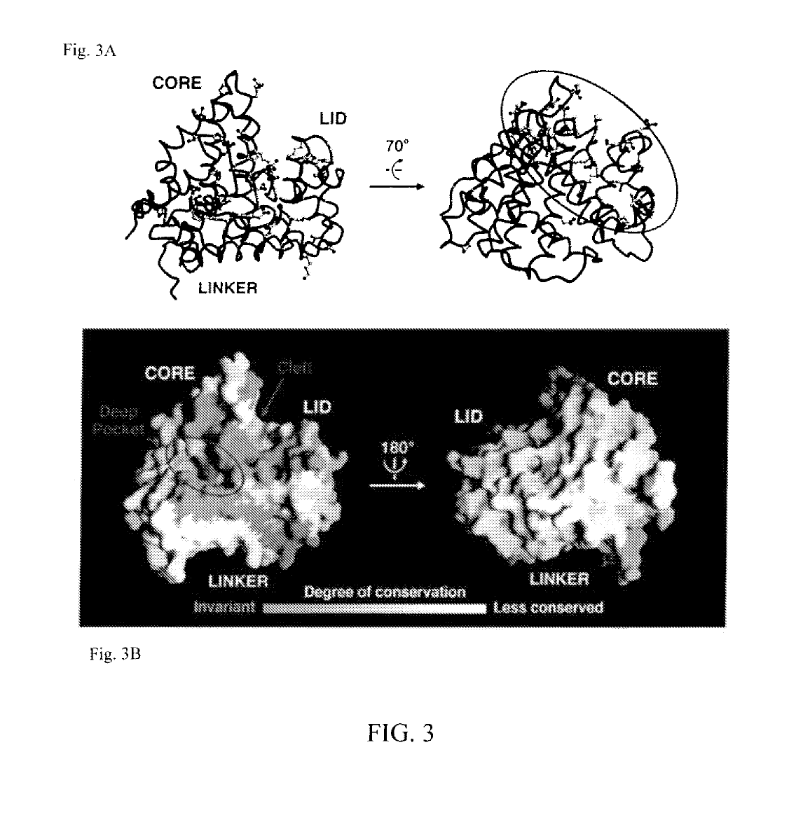

FIG. 3A shows the 39 invariant amino acids that are fully or partially exposed to solvent mapped onto the surface of the PTPA structure. The side chains of these amino acids are shown in yellow. As can he seen in FIG. 3A, most of the invariant and solvent-exposed residues map to one side of the PTPA structure circled in blue. FIG. 3B shows a surface map of PTPA with the invariant residues in green with invariant residues colored dark green, less conserved residues are colored light green, and non-conserved residues white. Invariant residues are primarily located in the surface patch that surrounds the deep pocket between the core and the linker domains (circled in red). This surface patch bifurcates into two additional areas, one at the interface between the linker and the lid domains, and the other at the bottom end of the cleft between the core and the lid domains. This analysis strongly suggests that the deep pocket plays an important role in PTPA function and that the contiguous surface patch is likely involved in binding to a protein partner such as the catalytic subunit of PP2A.

PTPA is thought to preferentially bind PP2A A-C dimmer, and because both PP2A and PTPA are highly conserved across species, a conserved set of amino acids may be involved in their interactions with each other. To identify PP2A-interacting elements on the surface of PTPA, 18 individual missense mutations of human PTPA were generated and purified to homogeneity. Each of the 18 mutations targets a surface amino acid that is invariant among the five PTPA homologs compared in FIG. 2, and all 18 mutant PTPA proteins appeared to be well-folded.

Recombinant PP2A A subunit was purified as a glutathione S-transferase (GST) fusion protein, and an N-terminal His₈ tagged PP2A C subunit was generated and purified using a Ni-NTA affinity column. The PP2A A and C subunits were then assembled into PP2A A-C dimers in vitro. The recombinant PP2A A-C dimer was shown to exhibit an identical level of phosphatase activity as PP2A A-C dimer purified from bovine brain.

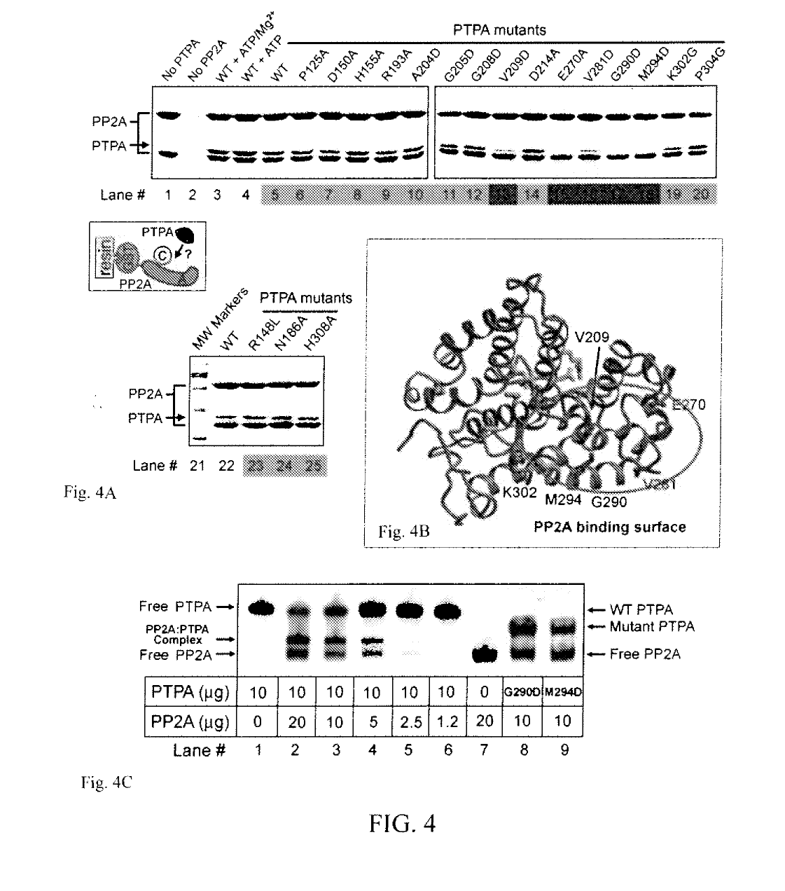

The interaction between PP2A and various PTPA mutant proteins was examined using a GST-mediated pull-down assay. FIG. 4A shows the results of this testing. The PP2A A-C dimer formed a stable complex with the wild type (WT) PTPA independent of ATP or magnesium ion (lanes 3-5). Twelve of the 18 PTPA mutations, including R148L, D150A, H155A, A204D, and G205D, did not have a significant effect on this interaction (lanes 6-12, 14 and 20). However, five mutations, V209D, E270A, V281D, G290D, and M294D, led to significantly compromised binding of PP2A (lanes 13 & 15-18) and a sixth mutation, K302G, also negatively affected binding to PP2A (FIG. 4A, lane 19). As shown in FIG. 4B, these six mutations all map to the same general area encompassing the border between the lid and linker domains (circled in purple). In FIG. 4B, the side chains of Val 209, Glu 270, Val 281, Gly 290, and Met 294 are shown in red and Lys 302 is shown in orange. These observations identify the lid-linker border area as the region of PTPA responsible for PP2A binding.

FIG. 4C shows native polyacrylamide gel electrophoresis (native-PAGE) used to further characterize the interaction with PP2A A-C dimer to PTPA. Under the experimental conditions used, the free A-C dimer migrated ahead of free WT PTPA on native-PAGE (lanes 1 and 7). The WT PTPA-PP2A complex migrated just behind the free A-C dimer (lanes 2-5). Quantification of the binding results gave rise to a dissociation constant of approximately 1 μM between PP2A A-C dimer and PTPA. The binding affinity reported here only serves as a reference, as it is likely to be influenced by cellular environments. Nonetheless, given the μM concentrations of PTPA in many tissues and organs, this binding affinity is in accord with biologically meaningful interaction in cells. Consistent with our earlier observations, two PTPA mutants, G290D and M294D, failed to form a detectable complex with PP2A (lanes 8 and 9). It should be noted that G290D and M294D migrated faster than WT PTPA on native PAGE due to one extra negative charge.

PTPA with PP2A A-C dimer have been shown to give rise to detectable ATPase activity when combined while neither component alone has any ATPase activity. FIG. 5A shows confirmation of these results as recombinant PP2A A-C dimer (open triangles) and PTPA (filled triangles) alone did not exhibit any detectable ATPase activity in the presence of 1 mM ATP and 5 mM Mg²⁺. However, incubation of PTPA with an equi-molar amount of PP2A A-C dimer yielded a significant ATPase activity (filled circles), with a catalytic turnover rate of approximately 2 per minute. FIG. 5A also shows that this activity can be inhibited by okadaic acid which is known to bind to the catalytic subunit of PP2A. FIG. 5B shows a kinetic analysis of the ATPase activity of the PTPA-PP2A A-C dimmer complex which yields a Km of about 0.4 mM. These results suggest that ATPase activity observed depends upon the interaction between PTPA and PP2A A-C dimer as the amount of PP2A in each reaction correlated with the levels ATPase activity. Our data demonstrate that PTPA and PP2A together may constitute a composite ATPase.

The ATPase activity of the 18 mutant PTPA proteins described above was examined in the presence of an equi-molar amount of PP2A A-C dimmer, and these results were compared to the ATPase activity of WT PTPA to identify amino acids important for the ATPase activity. FIG. 5C shows the results of this analysis. PAPA mutants, P125A, N186A, R193A, D214A, K302G, P304G, and H308A, exhibited a similar level of ATPase activity as WT PTPA. In contrast, 10 PTPA mutants, R148L, D150A, H155A, A204D, G205D, V209D, F270A, V281D, G290D, and M294D, exhibited significantly lower or undetectable ATPase activity.

FIG. 5D shows the location of the mutant amino acids effecting ATPase activity mapped to the surface of PTPA with inhibitory amino acid side chains in red. These mutations can be separated into two groups. One group including V209D, E270A, V281D, G290D, and M294D (circled in purple), showed both reduced ATPase activity, and compromised or undetectable binding to PP2A A-C dimer (see FIG. 4 for comparison) providing support that interaction between PTPA and A-C dimer is required for the ATPase activity. The second group including R148L, D150A, H155A, A204A, and G205D (circled in orange), showed reduced ATPase activity but maintained good interactions with PP2A (see FIG. 4 for comparison). The five residues in this group map to the area inside or close to the deep pocket between the core and the linker domains (circled in orange). These data demonstrate that PTPA binding by PP2A is necessary but not sufficient to stimulate the ATPase activity, and that a number of residues in the deep pocket of PTPA directly contribute to the ATPase activity.

The results described above suggest that ATP may bind to the deep pocket of PTPA. Therefore, human PTPA was crystallized in the presence of ATPγS, a non-hydrolyzable analog of ATP, and the structure for these co-crystals was determined by molecular replacement (Table 1). Eight molecules of PTPA were shown in each asymmetric unit, only four of which contain ATPγS. FIG. 5E shows a comparison of ATPγS-bound PTPA (green and light green) with nucleotide-free PTPA (blue and light blue). The overall structure of the ATPγS-bound PTPA is very similar to the nucleotide-free PTPA with 0.69 Å RMSD for all 301 aligned Cα atoms. The deep pocket is circled in magenta, and the amino acid side chains of wild type amino acids affected by mutation are shown in red. Binding of ATPγS to PTPA only appears to cause a conformational change to one region of PTPA, as the only region that exhibits a significant conformational change is residues 204-210, which are located next to the deep pocket (indicated by the blue arrow). It should be pointed out that the ATPγS binding site is in close proximity to the putative P-loop as suggested by Goris and colleagues (Cayla et al. J Biol Chem. Jun. 3, 1994;269(22): 15668-75).

The mode of phosphate binding indicated by the PTPA-ATPγS co-crystal structure does not resemble that of any known ATPase. FIG. 5F is a stereo diagram of bound ATPγS and the surrounding region or PTPA shown as a wire diagram colored green. The 2Fₒ-F_{c} electron density map, shown in 1.2σ of an ADP molecule derived from the ATPγS (shown in red) is contoured gray. The adenine and the ribose moieties of ATPγS bind within the deep pocket in close contact with residues Arg148, Asp150, His155, Ala204, and Gly205 whose side chains are provided in yellow. A mutation in each of these five amino acids led to compromised ATPase activity providing an explanation for the observed phenotype. In contrast to other known ATPases, the α and β phosphate groups of ATPγS are largely exposed to solvent. Consequently, the γ phosphate is flexible and disordered in the crystals and does not have well-defined electron density. Therefore, the γ-phosphate is not modeled in FIG. 5F. These observations suggest that the γ phosphate, and to a lesser extent the α and β phosphates, may be bound by PP2A and provide further evidence that the ATPase activity exhibited by PTPA may be a composite ATPase activity requiring binding of PP2A to PTPA. Therefore, PP2A may play an active role in binding to the phosphate groups of ATP and may provide catalytic residue that attack the leaving γ-phosphate atom. Additionally, ATP may enhance the interaction between PTPA and PP2A.

The effect of this composite ATPase activity was examined by observing the phosphotyrosyl phosphatase activity of PP2A A-C dimer in the presence and absence of an equi-molar amount of PTPA. Although no physiologically relevant phosphotyrosyl substrate has been identified yet, PTPA has been shown to be capable of stimulating the phosphotyrosyl phosphatase activity of PP2A A-C dimer using various phosphotyrosyl peptides including p-nitrophenylphosphate (pNPP) as substrate. FIG. 6A shows that the pNPP phosphatase (pNPPase) activity of PP2A A-C dimer is enhanced in the presence of PTPA in a concentration-dependent manner (filled triangles), and that this pNPPase activity requires ATP/Mg²⁺ (filled boxes). At equi-molar concentrations, PTPA-stimulated PP2A exhibited an approximately 6-fold higher pNPPase activity than PP2A in the absence of PTPA (filled triangles).

FIG. 6B shows that a non-hydrolyzable ATP analog, ATPγS, fails to stimulate the pNPPase activity of PP2A when provided to PP2A alone or in the presence of PTPA (far right columns) whereas PP2A pNPPase activity is greatly enhanced in the presence of PTPA and ATP/Mg²⁺ (middle columns). Control reactions of PP2A and PP2A with PTPA in the absence of ATP/Mg²⁺ are provided in the far left column. FIG. 6C shows that none of the PTPA mutants that exhibited compromised ATPase activity are able to enhance the pNPPase activity of PP2A. In particular, it is noted that the mutants of amino acids shown above are in close proximity to ATPγS bound to PTPA; specifically, R148L, D150A, H155A, A204D and G205D, do not stimulate the pNPPase activity of PP2A. These observations provide further support that the composite ATPase activity of the PP2A-PTPA complex is important for stimulating the phosphotyrosyl activity of PP2A A-C dimers.

FIG. 6G shows the effect of WT PTPA and various PTPA mutants on the pTyr activity of PP2A using a peptide substrate, a fluorogenic phosphotyrosine peptide (MCA-Gly-Asp-Ala-Glu-pTyr-Ala-Ala-Lys(DNP)-Arg-NH₂). The tyrosine phosphatase activity was monitored by chymotrypsin which only cleaves the dephosphorylated peptide at the carboxy end of Tyr. Cleavage of the peptide results in enhanced fluorescence intensity which was monitored at 328 nm (excitation) and 395 nm (emission). Consistent with previously described observations in the absence of either PTPA or ATP/Mg²⁺, only a basal level of phosphatase activity for the PP2A A-C dimer is observed (columns 1 and 2), and incubation of an equi-molar amount of WT PTPA and PP2A in the presence of ATP/Mg²⁺ resulted in enhanced phosphotyrosine phosphatase activity (column 3). PTPA mutants having compromised ATPase activity, R148L, D150A, H155A, A204D, G205D, V209D, F270A, V281D, G290D, and M294D, did not stimulate the pTyr activity of PP2A over basal levels (columns 5-7, 11-13 and 15-18) while PTPA mutants, P125A, N186A, R193A, D214A, K302G, P304G, and H308A exhibited similar stimulation of PP2A pTyr activity. These results complement the results obtained using pNPP as the substrate (compare FIGS. 6C and 6G).

The impact of PTPA on the phosphoserine (pSer) or phosphothreonine (pThr) phosphatase activity of PP2A was tested using phosphorylase b, a pSer substrate, which is converted to phosphorylase a by phosphorylase b kinase in vitro and has been shown to be active as a substrate for PP2A. FIG. 6D shows that in the absence of PTPA pSer, activity is observed (column 1), and that the presence of an equi-molar amount of WT PTPA leads to an approximately 45 percent reduction of PP2A pSer activity (column 2). Three PTPA mutants, D150A, A204D, and G205D, which retained binding to PP2A but had compromised ATPase activity, exhibited a similar inhibitory effect on the pSer activity of PP2A as the WT PTPA (columns 3-5). Another PTPA mutant, P304G, which retained both binding to PP2A and ATPase activity, reduced the PP2A pSer activity (column 7). In contrast, PTPA mutant, G290D, which was shown to have a compromised binding to PP2A A-C dimer, did not inhibit the PP2A pSer activity of PP2A (column 6). Additionally, it is noted that the presence of ATP/Mg²⁺ did not effect PP2A pSer activity (compare green to purple bars) suggesting that ATP hydrolysis is not required for the inhibitory effect of PTPA on the pSer activity of PP2A. These results suggest that binding of PTPA to PP2A A-C dimer may result in a conformation change in the C subunit of PP2A which inhibits pSer/pThr activity of PP2A.

FIG. 6E provides further evidence the pSer/pThr activity of PP2A is effected by PTPA by showing that PP2A activity on phosphorylase b is inversely correlated with the concentrations of PTPA. In FIG. 6E, PTPA was titrated against a fixed amount of PP2A A-C dimer (filled boxes), showing that WT PTPA inhibited the pSer activity of PP2A in a concentration-dependent manor, with maximal reduction occurring at about 20-to-1 molar ratio of PTPA to PP2A. At this molar ratio, the majority of the PP2A A-C dimer appears to be bound by PTPA resulting in PP2A pSer/pThr activity which was approximately 25% of the pSer activity of PP2A alone. Increasing PTPA concentrations beyond this molar ratio led to little further reduction of PP2A activity. In contrast, similar titrations carried out using PTPA mutant G290D, which shows compromised binding to PP2A, did not show an inhibitory effect on the pSer activity of PP2A (filled triangles).

These observations indicate that PTPA modulates the phosphatase activity of PP2A reducing pSer/pThr activity and enhancing pTyr activity and that PTPA may alter the relative specificity of PP2A, changing it from a pSer/pThr phosphatase to pTyr phosphatase. FIG. 6F shows that the substrate preference of PP2A A-C dimer shifts from phosphorylase b to pNPP in the presence of WT PTPA at a stoichiometric ratio of 1:1 by about 10-fold (compare column 1, no PTPA, to column 2, PTPA). This effect is not observed for PTPA mutants that exhibit either compromised ATP binding but good PP2A binding, A204D and G205D (columns 3 and 4), or PTPA mutants that exhibit compromised PP2A binding but good ATP binding, V209D and G290D (columns 5 and 6). However, this substrate's specificity of PP2A is not effected by a PTPA mutant that exhibited both good PP2A binding and good ATP binding, P304G (column 7).

Without wishing to be bound by theory, these results suggest that PTPA may alter the relative preference of PP2A for pSer/Thr and pTyr substrates by as much as 10-fold in an ATP hydrolysis-dependent manner. This modulation of PP2A may have major ramifications on the signaling specificity and outcome in cells.

Based on the results described above, the modulatory effect of PTPA on PP2A appears to occur in two steps as shown in FIG. 6H. First, PTPA binds to PP2A independently of ATP/Mg²⁺ (panel 1), reducing the pSer/pThr activity of PP2A. Second, the PTPA-PP2A complex binds (panel 2) and hydrolyzes ATP (panel 3) increasing the pTyr activity of PP2A. PTPA binding to PP2A and subsequent ATP hydrolysis are predicted to bring conformational changes in the C subunit of PP2A (panel 3). Although not well understood, this conformational change appears to be transient, stable only on the order of minutes, indicating that ATP-hydrolysis may activate a conformational change in PTPA and/or PP2A, and PTPA and/or PP2A may automatically switch back to its original conformation after several minutes. This is supported by evidence that removal of ATP/Mg²⁺ after incubation with PTPA-PP2A results in rapid reduction in pTyr activity (data not shown). The recently discovered peptidyl prolyl cis-trans isomerase activity of PTPA may provide an explanation for this conformational change. It was recently shown that the cis-trans isomerization of Xaa-Pro occurs in the time range of minutes, and that the isomerase activity of PTPA is stimulated by the presence of ATP/Mg²⁺.

In addition to the substrate specificity modulating activity of PTPA on PP2A, PTPA may compete for binding to the PP2A A-C dimer with the regulatory subunits (B subunits) of PP2A. Without wishing to be bound by theory, in the absence of PTPA or at low concentrations of PTPA, PP2A A-C dimer primarily acts as a pSer/pThr phosphatase and readily forms a holoenzyme with regulatory (B) subunits. However, in the presence of μM concentrations of PTPA, PTPA may compete for binding to PP2A with B subunits, not only leading to a decrease of PP2A's preference for the pSer/pThr substrates but also altering the substrate's specificity or cellular location of PP2A provided by the B subunit and further reducing PP2A's ability to act on various substrates. Binding and hydrolysis of ATP may further increase the binding affinity of PTPA or PP2A, further altering the substrate's specificity and cellular location specified by the B-subunits while increasing the preference of PP2A for pTyr substrates. In either case, PTPA may have a dramatic effect on substrate specificity and activity of PP2A and is likely to have a direct consequence on cellular function.

In various embodiments of the invention, the atomic coordinates of the deep pocket ATP binding site of PTPA may be used to design or identify molecules that specifically inhibit the ATPase activity of PTPA. Because the ATP binding pocket of PTPA is different from other known ATPases, agents that specifically bind to the ATP binding pocket of PTPA may selectively reduce the ATPase activity of PTPA. The “deep pocket” of PTPA as used herein, refers to the pocket on the surface of PTPA formed by β2, α6, β3, α9, and α17. In some embodiments of the invention, non-hydrolyzable compounds that are substantially complementary to the deep pocket of PTPA may inhibit the ATPase activity of PTPA, or the ATPase activity of a PTPA/PP2A complex and, therefore, may be considered ATPase inhibitors of PTPA. In various methods embodied herein, the atomic coordinates of the deep pocket may be used to electronically screen spatial coordinates of stored candidate compounds. In other embodiments, the atomic coordinates of the deep pocket may be used to design compounds that are substantially complementary to the pocket.

In still other embodiments, compounds may be identified or designed which mimic the shape, size, and/or charge of an ATP molecule, or a portion of an ATP molecule bound within the deep pocket of PTPA. For example, a PTPA inhibitor may have a three-dimensional structure corresponding to at least a portion of ATP bound to PTPA, or an inhibitor identified by applying a three-dimensional modeling algorithm to the atomic coordinates of ATPγS bound to PTPA and electronically screening stored spatial coordinates of candidate compounds against the atomic coordinates of ATPγS.

Candidate compounds that are identified as substantially complementary to the deep pocket, or designed to be substantially complementary to the deep pocket or compounds that mimic the structure of ATP bound to PTPA, may be synthesized using known techniques and tested for the ability to inhibit the ATPase activity of PTPA. Compounds that are found to effectively inhibit the ATPase activity of PTPA may be considered “ATPase inhibitors” for PTPA and may be used to modulate the activity of PTPA and PP2A. In still other embodiments, ATPase inhibitors of PTPA may be used as part of a pharmaceutical composition and administered to individuals in need thereof

The term “complementary” or “substantially complementary” compound as used herein, refers to a compound having a size, shape, charge or any combination of these characteristics that allow the compound to substantially fill the space created by β2, α6, β3, α9, and α17, or deep pocket, on the surface of PTPA. A compound that substantially fills the deep pocket without overlapping portions of elements β2, α6, β3, α9, and α17, even if various portions of the space remain unfilled, may be considered “substantially complementary.”

ATPase inhibitors, such as those described above, may be identified using methods of various embodiments of the invention. For example, a three-dimensional modeling algorithm may be used to determine the atomic coordinates of the deep pocket of PTPA or an ATP molecule bound within the deep pocket of PTPA, and these atomic coordinates may be used to electronically screen spatial coordinates of stored candidate compounds in order to identify compounds that may be substantially complementary to the deep pocket of PTPA, or mimic at least a portion of the structure of ATP bound to PTPA. Such compounds may be considered ATPase inhibitors of PTPA and may be tested for an ATPase inhibitory effect on PTPA.

As described above, ATP and non-hydrolyzable analogues of ATP bind within the deep pocket of PTPA in such a way that the α, β, and γ phosphates of the ATP or non-hydrolyzable analogue remain unbound. Therefore, in certain embodiments, compounds having a three-dimensional structure corresponding to the atomic coordinates of ATP bound within the deep pocket of PTPA may be ATPase inhibitors. In some such embodiments, ATPase inhibitors may be an ATP analogue or a non-hydrolyzable ATP. Additionally, because the α, β, and γ phosphates of ATP may not be required for ATP binding within the deep pocket of PTPA, in other embodiments, adenine, adenosine, adenine or adenosine analogues or mimetics of adenine or adenosine, may be ATPase inhibitors of PTPA. In still other embodiments, ATPase inhibitors may have a size, shape, charge or combination of these characteristics similar to ATP, adenine, adenosine, ATP analogues, non-hydrolyzable ATP, adenine or adenosine analogues or mimetics of adenine or adenosine. In still other embodiments, ATPase inhibitors of PTPA may have none of the characteristics of ATP or adenosine.

Any ATPase inhibitor identified using the techniques described herein, may bind to PTPA with at least about the same affinity as ATP, and in certain embodiments, the ATPase inhibitor may have an affinity for PTPA that is greater than the affinity of ATP for PTPA. Thus, such ATPase inhibitors my bind to PTPA and inhibit the ATPase activity required to perform the peptidyl-proline cis-trans isomerase on the catalytic subunit of PP2A, thereby providing methods and compounds for modulating the activity of PP2A. For example, without wishing to be bound by theory, inhibition of the ATPase activity of PTPA may reduce or inhibit phosphotyrosyl activity of PP2A or increase or enhance PP2A mediated Ser/Thr phosphorylation, and modulating the activity of PP2A may provide the basis for treatment of various cell cycle modulation or proliferative disorders including, for example, cancer and autoimmune disease.

Determination of the atomic coordinates of the deep pocket of PTPA may be carried out by any method known in the art. For example, the atomic coordinates provided in embodiments of the invention, or the atomic coordinates provided by other human PTPA crystallographic or NMR structures may be provided to a molecular modeling program and the pocket created by β2, α6, β3, α9, and α17, human amino acids, Arg148-Tyr151, Thr153-Lys168, Glu202-Gly205, Gln207-Trp210, and Phe303-Ile306, respectively, may be visualized. Because various PTPA proteins across species have a high level of sequence similarity (see FIG. 2) in other embodiments of the invention, atomic coordinates for elements corresponding to β2, α6, β3, α9, and α17 from other species, such as, for example, X. laevis PTPA, D. melanogaster PTPA, C. elegans PTPA, and S. cerevisae YPA1 and YPA2 shown in FIG. 2, may be used to model the structural features of the deep pocket of PTPA. In still other embodiments, two or more sets of atomic coordinates corresponding to β2, α6, β3, α9, and α17 in human PTPA may be compared and composite coordinates representing the average of these coordinates may be used to model the structural features of the deep pocket. The atomic coordinates used in such embodiments may be derived from purified PTPA alone or PTPA bound to PP2A, or another substrate protein, accessory protein, protein fragment or peptide with or without ATP, non-hydrolyzable ATP analogue, adenosine or adenosine analogue. In general, atomic coordinates defining a three-dimensional structure of a crystal of a PTPA that diffracts X-rays for the determination of atomic coordinates to a resolution of 5 Angstroms or better are preferred.

Having defined the structural features of the deep pocket of PTPA, mimetics or small molecules substantially complementary to the deep pocket may be designed. Various methods for molecular design are known in the art, and any of these may be used in embodiments of the invention. For example, in some embodiments, compounds may be specifically designed to fill an open area within the deep pocket left within the spatial coordinates defining elements β2, α6, β3, α9, and α17 or random compounds may be generated and compared to the spatial coordinates of the open area within the deep pocket. In other embodiments, stored spatial coordinates of candidate compounds contained within a database may be compared to the spatial coordinates of the open area within the deep pocket. In certain embodiments, molecular design may be carried out in combination with molecular modeling.

In particular embodiments of the invention, the atomic coordinates of an ATP bound within the deep pocket of PTPA, as provided herein, may be used as a basis for mimetic or small molecule ATPase inhibitor design. In such embodiments, compounds that mimic the structure of ATP bound to PTPA and maintain the molecular contacts, such as, for example, hydrogen bonds and van der Waals contacts, may be created. In general, compounds created in such embodiments that deviate from the atomic coordinates of the ATP bound by PTPA by a root mean square deviation of less than 10 angstroms are preferred. In other such embodiments, additional features may be added to an adenosine or non-hydrolyzable ATP backbone to create a new compound which provides improved contact between the deep pocket of PTPA and the compound. For example, such compounds may have improved or added hydrogen bonding or improved or added van der Waals contacts.

Methods for performing structural comparisons of atomic coordinates of molecules including those derived from protein crystallography are well known in the art, and any such method may be used in embodiments of the invention to either compare atomic coordinates of various PTPA structures or compare spatial coordinates of designed, random or stored candidate compounds. For example, structural comparisons may be carried out using a distance alignment matrix (DALI), Sequential Structure Alignment Program (SSAP), combinatorial extension (CE) or any such structural comparison algorithm.

Compounds identified using various methods of embodiments of the invention may be tested for binding to PTPA and/or to determine the compound's ability to inhibit ATPase activity of PTPA or modulate the activity of PP2A by, for example, testing for pTyr activity. Such testing may be carried out by any method, such as, for example, those described herein above. In general, such methods may include contacting PTPA with an identified compound and detecting binding to PP2A and/or ATPase activity. Such methods are well known in the art and may be carried out in vitro, in a cell-free assay, or in vivo, in a cell-culture assay. For example, radio-labeled ATP may be provided to purified PTPA and PP2A and ATP may be separated from hydrolyzed ADP by thin-layer chromatography (TLC) to determine a test for ATPase activity following a period of incubation with an agent identified to bind to PTPA. The compound may be considered an ATPase inhibitor if the compound binds to PTPA and inhibits its ATPase activity.

Embodiments of the invention also include pharmaceutical compositions including ATPase inhibitors that bind within the deep pocket of PTPA and inhibit ATPase activity of PTPA or are identified using methods of embodiments described herein above and a pharmaceutically acceptable carrier or excipient. Such pharmaceutical compositions may be administered to an individual in an effective amount to alleviate conditions associated with PP2A activity.

The invention described herein encompasses pharmaceutical compositions including a therapeutically effective amount of any of an ATPase inhibitor in dosage form and a pharmaceutically acceptable carrier, wherein the compound inhibits the activity of PTPA, thus inhibiting the phosphotyrosyl activity of PP2A. In another embodiment, such compositions include a therapeutically effective amount of an ATPase inhibitor in dosage form and a pharmaceutically acceptable carrier in combination with a chemotherapeutic and/or radiotherapy, wherein the ATPase inhibitor binds to PTPA and inhibits the activation of the phosphotyrosyl activity of PP2A, thus promoting apoptosis and enhancing the effectiveness of the chemotherapeutic and/or radiotherapy. In various embodiments of the invention, a therapeutic composition for modulating PP2A activity can be a therapeutically effective amount of an ATPase inhibitor that binds to PTPA.

Embodiments of the invention also include methods for treating a patient having a condition characterized by aberrant cell growth wherein administration of a therapeutically effective amount of an ATPase inhibitor is administered to the patient, and the ATPase inhibitor binds to PTPA inducing apoptosis within the area of the patient exhibiting aberrant cell growth. The method may further include the concurrent administration a chemotherapeutic agent, such as, but not limited to, alkylating agents, antimetabolites, anti-tumor antibiotics, taxanes, hormonal agents, monoclonal antibodies, glucocorticoids, mitotic inhibitors, topoisomerase I inhibitors, topoisomerase II inhibitors, immunomodulating agents, cellular growth factors, cytokines, and nonsteroidal anti-inflammatory compounds.

The ATPase inhibitors of the invention may be administered in an effective amount. An “effective amount” is an amount of a preparation that alone, or together with further doses, produces the desired response. This may involve only slowing the progression of the disease temporarily, although it may involve halting the progression of the disease permanently or delaying the onset of or preventing the disease or condition from occurring. This can be monitored by routine methods known and practiced in the art. Generally, doses of active compounds may be from about 0.01 mg/kg per day to about 1000 mg/kg per day, and in some embodiments, the dosage may be from 50-500 mg/kg. In various embodiments, the compounds of the invention may be administered intravenously, intramuscularly, or intradermally, and in one or several administrations per day. The administration of ATPase inhibitors can occur simultaneous with, subsequent to, or prior to chemotherapy or radiation.

In general, routine experimentation in clinical trials will determine specific ranges for optimal therapeutic effect for each therapeutic agent and each administrative protocol and administration to specific patients will be adjusted to within effective and safe ranges depending on the patient's condition and responsiveness to initial administrations. However, the ultimate administration protocol will be regulated according to the judgment of the attending clinician considering such factors as age, condition and size of the patient, the potency of the ATPase inhibitor administered, the duration of the treatment and the severity of the disease being treated. For example, a dosage regimen of an ATPase inhibitor to reduce cellular proliferation or induce apoptosis can be oral administration of from about 1 mg to about 2000 mg/day, preferably about 1 to about 1000 mg/day, more preferably about 50 to about 600 mg/day, in two to four divided doses. Intermittent therapy (e.g., one week out of three weeks or three out of four weeks) may also be used.

In the event that a response in a subject is insufficient at the initial doses applied, higher doses (or effectively higher doses by a different, more localized delivery route) may be employed to the extent that the patient's tolerance permits. Multiple doses per day are contemplated to achieve appropriate systemic levels of compounds. Generally, a maximum dose is used, that is, the highest safe dose according to sound medical judgment. However, an individual patient may insist upon a lower dose or tolerable dose for medical reasons, psychological reasons or for virtually any other reason.

Embodiments of the invention also include a method of treating a patient with cancer or an autoimmune disease by promoting apoptosis wherein administration of a therapeutically effective amount of ATPase inhibitors, and the ATPase inhibitor binds to PTPA inhibiting the activation of the phosphotyrosyl activity of PP2A. The method may further include concurrent administration of a chemotherapeutic agent including, but not limited to, alkylating agents, antimetabolites, anti-tumor antibiotics, taxanes, hormonal agents, monoclonal antibodies, glucocorticoids, mitotic inhibitors, topoisomerase I inhibitors, topoisomerase II inhibitors, immunomodulating agents, cellular growth factors, cytokines, and nonsteroidal anti-inflammatory compounds.

A variety of administration routes are available. The particular mode selected will depend upon the severity of the condition being treated and the dosage required for therapeutic efficacy. The methods of the invention may be practiced using any mode of administration that is medically acceptable, meaning any mode that produces effective levels of active compounds without causing clinically unacceptable adverse effects. Such modes of administration include, but are not limited to, oral, rectal, topical, nasal, intradermal, inhalation, intra-peritoneal, or parenteral routes. The term “parenteral” includes subcutaneous, intravenous, intramuscular, or infusion. Intravenous or intramuscular routes may be particularly suitable for purposes of the present invention.

In one aspect of the invention, an ATPase inhibitor as described herein, with or without additional biological or chemotherapeutic agents or radiotherapy, does not adversely affect normal tissues while sensitizing aberrantly dividing cells to the additional chemotherapeutic/radiation protocols. While not wishing to be bound by theory because the ATPase inhibitors specifically target PTPA, marked and adverse side effects may be minimized. In certain embodiments, the composition or method may be designed to allow sensitization of the cell to chemotherapeutic agents or radiation therapy by administering the ATPase inhibitor prior to chemotherapeutic or radiation therapy.

The term “pharmaceutically-acceptable carrier” as used herein, means one or more compatible solid or liquid fillers, diluents or encapsulating substances which are suitable for administration into a human. The term “carrier” or “excipient” denotes an organic or inorganic ingredient, natural or synthetic, with which the active ingredient is combined to facilitate the application. The components of the pharmaceutical compositions are also capable of being co-mingled with the molecules of the present invention and with each other, in a manner such that there is no interaction which would substantially impair the desired pharmaceutical efficacy.

The delivery systems of the invention are designed to include time-released, delayed release or sustained release delivery systems such that the delivering of the ATPase inhibitors occurs prior to, and with sufficient time, to cause sensitization of the site to be treated. An ATPase inhibitor may be used in conjunction with radiation and/or additional anti-cancer chemical agents. Such systems can avoid repeated administrations of the ATPase inhibitor compound, increasing convenience to the subject and the physician, and may be particularly suitable for certain compositions of the present invention.

Many types of release delivery systems are available and known to those of ordinary skill in the art including, but not limited to, polymer base systems, such as, poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems including, for example: lipids including sterols, such as cholesterol, cholesterol esters and fatty acids or neutral fats, such as mono-, di- and tri-glycerides; hydrogel release systems; sylastic systems; peptide based systems; wax coatings; compressed tablets using conventional binders and excipients; partially fused implants and the like. Specific examples include, but are not limited to: erosional systems in which the active compound is contained in a form within a matrix such as those described in U.S. Pat. Nos. 4,452,775, 4,667,014, 4,748,034, and 5,239,660 and diffusional systems in which an active component permeates at a controlled rate from a polymer, such as described in U.S. Pat. Nos. 3,832,253, and 3,854,480. In addition, pump-based hardware delivery systems can be used, some of which are adapted for implantation.

Use of a long-term sustained release implant may be desirable. Long-term release is used herein, and means that the implant is constructed and arranged to deliver therapeutic levels of the active ingredient for at least about 30 days, and preferably about 60 days. Long-term sustained release implants are well-known to those of ordinary skill in the art and include some of the release systems described above.

The pharmaceutical compositions may conveniently be presented in unit dosage form and may be prepared by any of the methods well known in the art of pharmacy. All methods include the step of bringing the active agent into association with a carrier that constitutes one or more accessory ingredients. In general, the compositions may be prepared by uniformly and intimately bringing the active compound into association with a liquid carrier, a finely divided solid carrier, or both and then, if necessary, shaping the product.

Compositions suitable for parenteral administration conveniently include a sterile aqueous preparation of an ATPase inhibitor which is preferably isotonic with the blood of the recipient. This aqueous preparation may be formulated according to known methods using suitable dispersing or wetting agents and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are water, Ringer's solution, and isotonic sodium chloride solution. In addition, sterile fixed oils are conventionally employed as a solvent or suspending medium. For this purpose, any bland fixed oil may be employed including synthetic mono- or di-glycerides. In addition, fatty acids, such as oleic acid, may be used in the preparation of injectables. Carrier formulation suitable for oral, subcutaneous, intravenous, intramuscular, etc. administrations can be found, for example, in Remington's Pharmaceutical Sciences, Mack Publishing Co., Easton, Pa. which is incorporated herein in its entirety by reference thereto.

### Experimental Procedures

### Protein Preparation

All constructs and point mutations were generated using a standard PCR-based cloning strategy. Full length PTPA (1-323), N-terminal 18-amino acid truncated PTPA (19-323), and all mutants with single point mutation were cloned into pET21b vector (Novagen), and overexpressed at room temperature in E. coli strain BL21(DE3) as C-terminally His₆-tagged proteins. The soluble fraction of the E. coli cell lysate was first purified by the Ni-NTA (Qiagen) affinity column to homogeneity and further purified by ion-exchange (Source 15Q, Amersham) and size exclusion (Superdex 200, Amersham) chromatography. The A subunit of PP2A was expressed as a GST fusion protein in pGEX-2T (Amersham) and was purified as described (Wu et al. J Biol Chem 276, 20688-20694). The C subunit of PP2A was cloned into baculovirus using the BacoluGold system (Pharmingen), with an N-terminal His8 tag, and expressed in suspension insect cell culture. The protein was purified as described above.

### Crystallization and Data Collection of PTPA

Crystals of N-terminal truncated PTPA (19-323) were grown by the hanging-drop vapor-diffusion method by mixing the protein (˜10 mg/ml) with an equal volume of reservoir solution containing 0.2 mM Mg Formate, 5% glycerol, 17.5% PEG3350 (w/v), and 0.1 M Bistris pH 6.8. Crystals appeared overnight and grew to full-size within three days. The crystals belong to the space group C2, with a=158.25 Å, b=43.79 Å, c=75.57 Å, and β=108.83°. There are two molecules per asymmetric unit. For ATPγS complex, purified PTPA protein was incubated with 1 mM ATPγS and 40 mM MgCl₂. Small clustered crystals were generated under conditions 0.2 mM Na Formate, 5% glycerol, 16% PEG3350 (w/v), 50 mM Glycine, 0.1 M BisTris pH 6.8. These crystals were extremely fragile in all cryo-protecting conditions tested and diffracted X-rays poorly. The diffraction spots of the PTPA-ATPγS crystals were split and integration was a challenge. These crystals belong to the space group P1, with a 80.04 Å, b=86.10 Å, c=95.10 Å, α=73.91°, β=89.97°, and γ=73.31°. There are 8 molecules in each asymmetric unit. Crystals were equilibrated in a cryoprotectant buffer containing reservoir buffer plus 20% glycerol (v/v) and were flash frozen in a cold nitrogen stream at −170° C. The native set was collected at NSLS beamline X-29 and processed using the software Denzo and Scalepack.

### Structure Determination

The structure of human PTPA was determined using the program PHASER and the atomic coordinates recently released in PDB (accession code 2G62). The model was built using O and refined using CNS. There are two molecules of PTPA in each asymmetric unit. NCS constraint was used in the early refinement cycles. The final refined atomic model contains amino acids 21-321 for one molecule and residues 21-203 and 207-321 for the other molecule.

### ATPase Activity Assay

Protein samples were prepared in 100 mM NaCl, 20 mM HEPES, pH 7.5, and 2 mM dithiothreitol. The release of free phosphate group was measured by the calorimetric Malachite Green Reagent (BioAssay Systems, Hayward, Calif.) using NaH₂PO₄ as phosphate standard. The protein concentration was determined by a combination of OD absorption at 280 nm and Bio-Rad Protein Assay (Bio-Rad).

pNPPase Activity Assay

The phosphatase activity using pNPP as substrate was performed on a 96-well plate. Briefly, 50 μl of 50 mM pNPP dissolved in 50 mM Tris-HCl (pH 8.2) buffer containing 1 mM DTT, 10 mM MgCl₂ and 0.1 mg/ml BSA was added to each well. The reaction was initiated by addition of 10 μl of PP2A samples in the presence or absence of ATP and PTPA. The plate was incubated at 37° C. for 5-20 minutes and the enzymatic reaction product was measured by the absorbance at 405 nm using a plate reader.

### Phosphotyrosine Phosphatase Assay Using a Peptide as the Substrate

A protein tyrosine phosphatase assay kit (EMD Biosciences) was used to measure phosphotyrosine phosphatase activity. 50 nM PP2A and PTPA, or PTPA mutants were added to a fluorogenic phosphopeptide substrate (MCA-Gly-Asp-Ala-Glu-pTyr-Ala-Ala-Lys(DNP)-Arg-NH₂, 1 μM) and incubated at room temperature under very low light conditions. After 10 minutes of incubation, sodium vanadate solution was added to stop the reaction. Subsequent incubation with chymotrypsin for 5 minutes resulted in the cleavage of only dephosphorylated peptide, which resulted in enhancement of the fluorescence intensity. The fluorescent intensity was monitored at 328 nm (excitation) and 395 nm (emission), using a Hitachi F2500 fluorescence spectrophotometer.

### Phosphorylase a Phosphatase Assay

To measure the phosphatase activity of PP2A, 5 μl of each PP2A sample was added to 45 μl of 0.5 mg/ml of radio-labeled ³²P-phosphorylase b dissolved in 50 mM MOPS buffer containing 1 mM DTT and 5 mM caffeine. Reactions were performed at 37° C. for 10 minutes, and stopped by addition of 10% TCA and 0.5 mg/ml BSA. Samples were kept on ice for 10 minutes to allow complete precipitation of proteins, and were centrifuged for 10 minutes at 14K rpm. Supernatants containing the released ³²P were added to 3 ml Ecosint and the intensity was measured by scintillation counter.

### Native Polyacrylamide Gel Electrophoresis (Native PAGE)

All assays were performed at 4° C. to offset heat generated by electrophoresis. 6% poly-acrylamide (37.5:1 acrylamide:bis-acrylamide) gels were used under the buffer condition of 65 mM Tris, pH 8.5, and 65 mM boric acid. All protein samples were prepared in a 40 μl volume containing 25 mM Tris, pH 8.0, 150 mM NaCl, 2 mM DTT, and 5% glycerol. After pre-running the gels for 15 minutes, half of each sample (20 μl) was loaded into each lane and was subjected to electrophoresis with constant electric field of 15 volts/cm. The gels were stained using coomassie blue, destained, and dried for photography. The gels were scanned by a densitometer, which allowed the estimate of the binding affinity between WT PTPA and PP2A A-C dimer.

### GST-Mediated Pull-Down Assay

Approximately 30 μg of stoichiometric PP2A A-C dimer were bound to 30 μl of glutathione resin via GST-A protein. The resin was washed with 200 μl assay buffer 3 times to remove excess unbound PP2A. Then 10 μg of WT or mutant PTPA were allowed to bind the resin in a 125-μl volume. After washing 4 times with an assay buffer containing 25 mM Tris, pH 8.0, 150 mM NaCl, and 2 mM dithiothreitol (DTT), the remaining protein and resin were mixed with 15-μl SDS sample buffer and applied to SDS-PAGE. The results were visualized by SDS-polyacrylamide gel electrophoresis (SDS-PAGE) with coomassie staining.

The present invention is not limited to the embodiments described and exemplified above, but is capable of variation and modification within the scope of the appended claims.

What is claimed is:

1. A PTPA binding compound comprising a molecule having a three-dimensional structure corresponding to the atomic coordinates of at least a portion of ATPγS bound to PTPA.

2. The compound of claim 1, wherein the molecule is an ATPase inhibitor of PTPA.

3. The compound of claim 1, wherein the molecule binds to PTPA.

4. The compound of claim 1, wherein the molecule binds to a pocket formed by β2, α6, β3, α9 and α17 of PTPA.

5. The compound of claim 1, wherein the molecule is a non-hydrolyzable ATP analogue.

6. The compound of claim 1, wherein the molecule comprises adenine and ribose moieties of ATP.

7. The compound of claim 1, wherein the molecule is selected from mimetics and analogs of adenosine.

8. The compound of claim 1, wherein the molecule is selected from mimetics and analogs of adenine.

9. The compound of claim 1, wherein the molecule binds to PTPA with a greater affinity than ATP.

10. The compound of claim 1, wherein the molecule inhibits modulation of PP2A by PTPA.

11. The compound of claim 1, wherein the molecule inhibits tyrosine phosphorylation catalyzed by PP2A.

12. The compound of claim 1, further comprising a pharmaceutically acceptable excipient or carrier.

13. A pharmaceutical composition comprising:
an effective amount of a compound that mimics the structure of at least a portion of ATPγS bound to PTPA; and
a pharmaceutically acceptable excipient or carrier.

14. A method for preparing a PTPA binding compound comprising:
applying a three-dimensional molecular modeling algorithm to the atomic coordinates of ATPγS bound to PTPA;
determining spatial coordinates of the ATPγS;
electronically screening stored spatial coordinates of candidate compounds against the spatial coordinates of the ATPγS; and
identifying compounds that mimic the structure of the ATPγS bound to PTPA.

15. The method of claim 14, further comprising identifying candidate compounds that deviate from the atomic coordinates of the ATPγS bound by PTPA by a root mean square deviation of less than about 10 angstroms.

16. The method of claim 14, further comprising testing the identified compounds for binding PTPA.

17. The method of claim 14, further comprising testing the identified compounds for binding to a pocket formed by β2, α6, β3, α9 and α17 of PTPA.

18. The method of claim 14, further comprising testing the identified compound for inhibiting ATPase activity of PTPA in the presence of PP2A.

19. The method of claim 14, further comprising identifying a compound that binds to PTPA with a greater affinity than ATP.

20. The method of claim 14, further comprising identifying compounds that inhibit modulation of PP2A by PTPA.

21. The method of claim 14, further comprising identifying compounds that inhibit PTPA stimulated tyrosine phosphorylation activity catalyzed by PP2A.

22. A pharmaceutical composition comprising:
an effective amount of a compound prepared by the method comprising:
applying a three-dimensional molecular modeling algorithm to the atomic coordinates of ATPγS bound to PTPA;
determining spatial coordinates of the ATPγS;
electronically screening stored spatial coordinates of candidate compounds against the spatial coordinates of the ATPγS; and
identifying compounds that mimic the structure of the ATPγS bound to PTPA; and
a pharmaceutically effective excipient or carrier.

23. A compound comprising a molecule substantially complementary to a pocket formed by β2, α6, β3, α9 and α17 of PTPA.

24. The compound of claim 23, wherein the molecule has a shape, a charge distribution, a size or combinations thereof substantially complementary to the pocket.

25. The compound of claim 23, wherein the molecule binds to PTPA.

26. The compound of claim 23, wherein the molecule binds to a pocket formed by β2, α6, β3, α9 and α17 of PTPA.

27. The compound of claim 23, wherein the molecule is an ATPase inhibitor of PTPA.

28. The compound of claim 23, wherein the molecule is a non-hydrolyzable ATP analogue.

29. The compound of claim 23, wherein the molecule comprises adenine and ribose moieties of ATP.

30. The compound of claim 23, wherein the molecule is selected from mimetics and analogs of adenosine.

31. The compound of claim 23, wherein the molecule is selected from mimetics and analogs of adenine.

32. The compound of claim 23, wherein the molecule binds to PTPA with a greater affinity than ATP.

33. The compound of claim 23, wherein the molecule inhibits modulation of PP2A by PTPA.

34. The compound of claim 23, wherein the molecule inhibits tyrosine phosphorylation catalyzed by PP2A.

35. The compound of claim 23, further comprising a pharmaceutically acceptable excipient or carrier.

36. A method for preparing a PTPA binding compound comprising:
applying a three-dimensional molecular modeling algorithm to the atomic coordinates of PTPA;
determining spatial coordinates of a pocket formed by β2, α6, β3, α9 and α17 of PTPA;
electronically screening stored spatial coordinates of candidate compounds against the spatial coordinates of the pocket; and
identifying compounds that are substantially complementary to the pocket.

37. The method of claim 36, further comprising testing the identified compounds for binding PTPA.

38. The method of claim 36, further comprising testing the identified compounds for binding to a pocket formed by β2, α6, β3, α9 and α17 of PTPA.

39. The method of claim 36, further comprising testing the identified compound for inhibiting ATPase activity of PTPA in the presence of PP2A.

40. The method of claim 36, further comprising identifying compounds that bind to PTPA with a greater affinity than ATP.

41. The method of claim 36, further comprising identifying compounds that inhibit modulation of PP2A by PTPA.

42. The method of claim 36, further comprising identifying compounds that inhibit PTPA stimulated tyrosine phosphorylation activity catalyzed by PP2A.
